(19) United States
(12) Patent Application Publication
(10) Pub. No.: US 2025/0325364 A1
(43) Pub. Date: Oct. 23, 2025

(54) VALVE PROSTHESIS WITH A PIVOTABLE RETENTION MEMBER

(71) Applicant: INNOVHEART S.r.l., Milano (MI) (IT)

(72) Inventors: Giovanni RIGHINI, Gland (CH); Gianluca PANE, Ivrea (TO) (IT); Enrico BRESCIANO, Milano (MI) (IT)

(21) Appl. No.: 18/866,737

(22) PCT Filed: May 26, 2023
(86) PCT No.: PCT/IB2023/055407

§ 371 Date: Nov. 18, 2024

(30) Foreign Application Priority Data
May 26, 2022 (IT) 102022000011042
May 26, 2022 (IT) 102022000011051

(51) Int. Cl. A61F 2/24

(52) U.S. Cl. CPC A61F 2/2418; A61F 2/2427; A61F 2220/0008; A61F 2220/0091

(57) ABSTRACT

A valve prosthesis for a heart valve includes a prosthetic member and a retention member to partially fix the prosthetic member with respect to a portion of heart tissue in an implanted condition of the valve prosthesis. The retention member has at least one component having a main portion is joined at an end thereof to at least one connection element via a pivotable connection for connecting the retention member to the prosthetic member. The connection element and the main portion are pivotable with respect to each other from a first position, with a minimal spatial requirement compatible with the passage thereof in a catheter, to at least one second position in the implanted condition of the prosthesis. Connection members stably connect the main portion and the connection element in the second position to limit stresses to which the flexible section is subjected under operating conditions.

### FIELD OF THE INVENTION

The present invention relates to the field of valve prostheses for cardiovascular valves.

The invention has been developed with particular regard, though in a non-exclusive manner, for a valve prosthesis for an atrioventricular heart valve, that is to say, the mitral valve or the tricuspid valve, which allows the blood to flow from the atrium to the ventricle in diastole and prevents the reflux thereof in the reverse direction in systole.

More specifically, the invention relates to valve prostheses which can be implanted with transcatheter percutaneous techniques.

The invention has been developed with particular regard for a valve prosthesis which is provided with a pivotable retention member.

### TECHNOLOGICAL BACKGROUND

Prostheses are artificial devices which are commonly used in order to replace or at least integrate the functionality of a portion of the member which is missing or damaged once it is implanted in an orthotopic position, that is to say, arranged in the natural seat of this portion of the member. The heart valve prostheses are used to make the flow of the blood in the heart unidirectional. Valve prostheses for atrioventricular heart valves, that is to say, for the mitral valve or the tricuspid valve, are used to regulate the flow of blood from the atrium to the ventricle of the heart in diastole, preventing the reflux thereof in the reverse direction, from the ventricle to the atrium in the heart, in systole.

Heart valves can be subject to malfunctions which inhibit the correct operation thereof; typically, the main malfunctions of the heart valves are constituted by valve regurgitation as a result of an incomplete closure thereof (for example, as a result of prolapse of the annulus, which causes a lack of coaptation between the valve leaflets) and stenosis which restricts the corresponding orifice (for example, as a result of calcification of the annulus). These malfunctions can be generated by a great variety of pathologies, which concern the same heart valve and/or the entire heart (for example, degenerative diseases, ischaemia, cardiomyopathies, congenital anomalies, and so on). In this case, if it is not possible to cure the malfunction via the pharmacological route or by means of surgical repair of the heart valve, a corresponding replacement prosthesis is implanted in the heart.

The implantation of a prosthesis for a heart valve is a rather complex intervention. In place of open-heart implantation procedures of the cardiovascular prostheses, which are particularly invasive and often require the interruption of the heartbeat and the extra-corporeal circulation of the blood, percutaneous transcatheter techniques are preferred, wherever possible and advisable, wherein the prosthesis is implanted in the heart with transluminal access through a peripheral inlet with low invasiveness, for example, starting from the femoral arteries. To this end, the prosthesis is initially compressed radially. A guiding system or delivery system which generally comprises one or more catheters guides the prosthesis as far as the implantation site in the heart. Once the prosthesis has been correctly positioned, it is released by the guiding system and expands, for example, as a result of resilient return or insufflation of a balloon which is positioned therein, so as to recover the operating dimensions thereof.

There have been proposed cardiovascular prostheses which comprise a prosthetic member which supports the prosthetic leaflets and a retention member which at least partially surrounds the prosthetic member in order to fix the prosthesis to a portion of heart tissue. Some of these prostheses comprise a retention member which cooperates with the prosthetic member in order to entrap the native leaflets of the heart valve and thereby to ensure the secure anchoring of the prosthesis. One of these prostheses is described in WO 2012/063228 and WO 2014/080339.

There is known a valve prosthesis in which the retention member is fixed to the prosthetic member when the prosthetic member is still in the radially compressed configuration before it expands into the operating configuration. Such a prosthesis is described in WO 2015/118464. In one embodiment described in this document, the retention member of the prosthesis is constructed by means of sub-components, each of which has articulation mechanisms which allow the resilient deformation thereof until taking up a configuration with a minimal radial spatial requirement. In this manner, the introduction and the deployment of the components of the retention member at the implantation site can be brought about inside catheters with small diameters which make the procedure safer and involve minimal invasiveness. More specifically, each sub-component comprises a central portion which is joined to two end portions with the interposition of a transition zone which acts as a unidirectional articulation, allowing in one direction the re-alignment of the ends in the same plane of the remaining portion of the sub-component, but preventing in the opposite direction a greater deflection through a predefined angle between the main plane of the sub-component and the axis of the prosthetic member, once the sub-component has been re-connected thereto.

A transseptal implantation procedure which uses such a prosthesis is described, for example, in WO 2021/014400.

The structure of the retention member of the prosthesis described in WO 2015/118464 has been found to be particularly effective because it avoids the risk of excessive deflection of the segments of the retention member (or containment portion) towards the interior of the ventricular chamber. In this manner, there are ensured both the continuity of contact of the retention member on the annulus of the native valve and the correct mutual alignment between the retention member and the central member of the prosthesis when the prosthesis is finally released. However, when the prosthesis is implanted, the transition zone which acts as an articulation or hinge between the central portion and each of the two ends of the sub-component is constantly loaded by a variable load which is also associated with the heartbeat, with a resultant generation of a state of cyclical stress which requires that the transition zone meet specific requirements in terms of fatigue resistance for the high number of cycles which are applied over the years of expected operation of the prosthesis. Therefore, the need has arisen to develop improved solutions with respect to the one described in WO 2015/118464 so as to allow the same functionality of the retention member and in particular the sub-components thereof, but which at the same time ensure a greater resistance to the fatigue stresses in order to improve the performance levels and reliability thereof.

### STATEMENT OF INVENTION

An object of the invention is to overcome the disadvantages of the prior art via a valve prosthesis for a heart valve which is resistant and durable with respect to the cyclical stresses as a result of the heart activity.

Another object of the invention is to provide a valve prosthesis with a high level of durability and reliability over time.

Another object is to provide a heart valve with a retention member which ensures effective anchoring and retention thereof in the desired position.

Another object is to provide a heart valve with a retention member which is at the same time resistant, reliable and durable, with particular reference to the fatigue stresses with the high number of cycles resulting from the heartbeat and the thrusts applied to the prosthesis by the blood flow resulting from the opening and closing of the prosthetic valve leaflets.

Another object of the invention is to provide a retention member for a prosthesis of a heart valve which is simple to install with a transcatheter procedure with particular reference to the positioning thereof at an atrioventricular valve, such as the mitral valve or the tricuspid valve.

Another object of the invention is to provide a retention member for a heart valve which is easy to manufacture and to assemble and simple to use.

These objects and other objects and advantages are achieved by the present invention as defined in the appended claims.

According to a first aspect, there is described a valve prosthesis for a heart valve. The valve prosthesis comprises a prosthetic member which supports prosthetic leaflets which are able to reproduce the functionality of the valve leaflets of a native heart valve. The valve prosthesis comprises a retention member which is intended to at least partially fix the prosthetic member with respect to a portion of heart tissue in an implanted condition of the prosthesis. In this condition, the prosthesis is subjected to continuous stresses resulting from the heartbeat which brings about in particular the opening and closing of the leaflets of the valve in order to make the blood flow unidirectional.

The retention member described may comprise one or more components. Each component may have at least one connection element. By means of one or more of these connection elements, the retention member may be connected to the prosthetic member at least in the implanted condition of the valve prosthesis. Each component of the retention member may have at least one main portion, which is joined at one end thereof to at least one connection element via a pivotable connection, in order to connect the retention member to the prosthetic member. It is further described how the pivotable connection between the connection element and the main portion of the component allows the connection element to be orientated angularly with respect to the main portion of the component of the retention member.

More specifically, it is described how this pivotable connection defines an articulation or hinge between the connection element and the main portion of the component. The articulation may allow the connection element to move, particularly by pivoting with respect to the main portion, in order to move from a first position to at least one second position. The first position may be defined, for example, as the position in which the connection element and the main portion of the component take up a configuration with a minimal spatial requirement which is compatible with the passage thereof in a catheter. The term “minimal spatial requirement” is normally intended to be understood to mean the transverse spatial requirement with respect to the sliding direction in this catheter or more generally in the delivery device used in a transcatheter implantation procedure. A configuration with a minimal spatial requirement which is particularly effective is one in which the connection element is substantially aligned with the corresponding end of the main portion, that is to say, the configuration in which the longitudinal axis of the connection element is substantially coincident with the longitudinal axis of the main portion at the connection end thereof with respect to the connection element itself.

The second position may be the position which is taken up by the retention member in the implanted condition of the prosthesis or more generally a position near the implanted condition of the prosthesis. This second position may sometimes, but not necessarily, coincide with the position of maximum deflection of the articulation, that is to say, the limit angular position as far as which the connection element can be deflected with respect to the end of the main portion and which cannot be exceeded. In other words, the extent of movement of the articulation defined between the connection element and the end of the main portion from the initial position with a minimal spatial requirement, preferably, but not necessarily, aligned, locates a maximum limit which is determined and predefined and which is functional for the correct implantation of the prosthesis.

In the above-mentioned second position taken up by the retention member in the implanted condition of the prosthesis, the connection element is deflected with respect to the corresponding end of the main portion, for example, but in a non-limiting manner, orientated substantially transversely with respect thereto. The articulation may be configured in such a manner that at least in this second position the connection element is substantially blocked in relation to the main portion, preferably with an integral connection, which may in some cases be a connection with mechanical connection or interlocking connection. In this regard, it is described how the component of the retention member may comprise connection members in order to stably connect the main portion and the connection element at least in the above-mentioned second position so as to limit the stresses to which the flexible section is subjected under operating conditions of the valve prosthesis. The connection members serve to generate additional mechanical connections between the main portion and the connection element in the implanted position which may be a position near or substantially coincident with the position of maximum deflection of the connection element with respect to the main portion. This solution achieves a drastic improvement with respect to the prior art because, in this manner, the pivoting system of the connection element is reinforced so as to reduce the stresses on the flexible section of the pivoting connection which acts as a hinge and which is therefore provided to be relatively resilient or flexible in the position taken up by the component of the retention member in the implanted configuration of the valve prosthesis. Advantageously, this brings about a configuration which is structurally stable and robust for the transition zone which acts as a hinge or articulation of the retention member in the implanted position so as to ensure the requirement thereof involving solidity and resistance to the cyclical stresses resulting from the cardiac activity, substantially extending the duration thereof and therefore the reliability over time of the entire valve prosthesis.

The articulation can be configured in such a manner that, if the connection element is substantially blocked, in the second position in relation to the main portion, for example, by means of the above-mentioned integral connection, which as mentioned can be in some cases a connection with a mechanical connection or an interlocking connection, the blocking may be able to be unblocked with a manoeuvre carried out on the retention member, on one or more of the components thereof, on one or more of the main portions of the components and/or on one or more of the connection elements, for example, following a rotation and/or a predetermined thrust which is applied to one or more thereof, for example, during the positioning procedure of the retention member or more generally implantation procedure of the valve prosthesis. Additionally or alternatively, the articulation may be configured in such a manner that, in order to block the connection element, it is necessary to apply a rotation and/or a predetermined thrust to the retention member, one or more of the components thereof, one or more of the main portions of the components and/or one or more of the connection elements. These characteristics leave a specific freedom for manoeuvre for correctly positioning the retention member during the implantation procedure, allowing the articulation to be blocked in the definitive implanted position only when the correct positioning thereof has been confirmed.

According to a particular aspect, which is not necessarily subordinate to what has been set out above, it is also described that the pivotable connection which defines the articulation between the connection element and the main portion may comprise at least one flexible section. The flexible section thereby maintains the physical continuity between the main portion and the connection element. According to a particular aspect, the flexible section is arranged at the extrados of the articulation, that is to say, at the zone of the component of the retention member which takes up a greater radius of curvature when the articulation is bent and the connection element is deflected. In this manner, the physical continuity between the main portion and the connection element is located in the zone in which it most contributes to the solidity of the articulation and therefore to the reliability and resistance over time.

According to a particular aspect, which is not necessarily subordinate to one or more of the aspects indicated above, there is also described the possibility of providing the retention member in such a manner that the flexible section which can define the articulation between the connection element and the corresponding end of the main portion of the component comprises a portion with less resistance and a portion with greater resistance. The portion with less resistance may provide a predetermined flexibility for the pivotable connection during the movement from the first position with a minimal spatial requirement of the connection element to the second implanted position and/or the position with maximum deflection. In this manner, it is easy to bend the articulation in order to bring, for example, the connection element from the configuration, in which it is arranged substantially in continuation of the end of the main portion, to the configuration, in which it is, for example, transverse relative thereto. The portion with greater resistance may instead define or contribute to defining the resistance of the joint to the stresses to which it is subjected in the implanted condition of the valve prosthesis.

According to a particular aspect, it is described how the flexible section which can define the articulation can be in the form of a flexible plate, with a first portion having a first cross-section and a second portion having a second cross-section which is greater than the first cross-section, defining the portions with less resistance and greater resistance, respectively. The different cross-sections can readily be constructed with precision by means of cuts which are carried out in the structure of the component of the retention member.

According to another particular aspect, it is described how the flexible section may be non-extensible. In particular, the flexible section may be constructed integrally with the component of the retention member so as to form a single piece with the main portion and the connection element.

According to a particular aspect, which is not necessarily subordinate to one or more of the aspects indicated above, the connection members may comprise a system of wings which project laterally from the flexible section. The wings can be configured in order to engage in at least one corresponding cavity which is formed in the connection element. The wings can be supported in the cavity so as to generate in the implanted configuration an additional mechanical connection of the connection element with respect to the main portion which protects and in practice excludes the deformable and fragile portion of the pivotable connection, particularly the flexible section, from the exchange of cyclical loads between the elements of the annular structure.

According to another particular aspect, which is not necessarily subordinate to one or more of the aspects indicated above, it is described how the connection element can pivot resiliently. In particular it is described how the connection element can be resiliently connected to the main portion of the component of the retention member. The resilient pivoting is such that the retention member is resiliently urged towards the first position when it is moved away from it. The resilient pivoting provides a specific feedback during the positioning of the valve prosthesis, when the retention member is resistant to the surrounding heart tissue, for example, in the region of the annulus. The resilient pivoting can be blocked when the connection members intervene, for example, between the main portion and the connection element in the implanted position of the valve prosthesis, which block the connection element in such a manner that it cannot pivot with respect to the main portion any more.

According to another particular aspect, which is not necessarily subordinate to one or more of the aspects indicated above, the pivotable connection of the retention member can be constructed by means of cuts and apertures in a tubular element. In this manner, the structural continuity is advantageously maintained between the connection element and the main portion, also allowing the use of reliable processing technologies based on laser cutting and the generation of a production process which is generally simple and economical. Preferably, the tubular element can completely define the component, with a resultant economy and ease of production.

### BRIEF DESCRIPTION OF THE DRAWINGS

Additional features and advantages will be appreciated from the following detailed description of a preferred embodiment with reference to the appended drawings which are provided purely by way of non-limiting example and in which:

FIG. 1 is a schematic view of a valve prosthesis for a heart valve in the configuration which it can take up when it is implanted, and wherein for the sake of simplicity of illustration there are not illustrated the native leaflets or other cardiac structures, comprising a prosthetic member which is connected to a retention member with two components;

FIG. 2 is a perspective view of a component of a retention member which can be used in the valve prosthesis of FIG. 1, incorporating aspects of the present invention;

FIG. 3 is a detailed perspective view of a pivotable connection which defines an articulation of the component of FIG. 2, in a first position;

FIG. 4 is a view similar to the view of FIG. 3, in which the articulation of the component. is in a second position;

FIG. 5 is a longitudinal section of the articulation of FIG. 3;

FIG. 6 is a plan view of the articulation of FIGS. 3 to 5 in a third position;

FIG. 7 is a side view of the articulation of FIG. 6;

FIG. 8 is a bottom view of the articulation of FIG. 6;

FIG. 9 is a plan view of a first variant of the articulation of the component of the retention member;

FIG. 10 is a side view of the first variant of the articulation of FIG. 9;

FIG. 11 is a plan view of a second variant of the articulation of the component of the retention member;

FIG. 12 is a side view of the second variant of the articulation of FIG. 11;

FIG. 13 is a plan view of a third variant of the articulation for the component of the retention member;

FIG. 14 is a side view of the third variant of the articulation of FIG. 13;

FIG. 15 is a perspective view of a fourth variant of the articulation of the component of the retention member; and

FIG. 16 is a side view of the third variant of the articulation of FIG. 15.

### DETAILED DESCRIPTION

In the following embodiments, there are described characteristics which allow the invention to be carried out. The characteristics described can be combined with each other in various manners and are not necessarily limited to the precise embodiment to which the drawings and the relevant description refer. In other words, a person skilled in the art who reads the following description will know how to obtain the useful pieces of information for knowing the way to carry out one or more of the features described by combining it with one or more of the other features described without the particular formulation of the description, the paragraphs, the phrases or the drawings constituting a limitation on the possibility of isolating one or more of the features described and illustrated in order to combine them with one or more of any of the other features described and illustrated. In greater detail, in the present description there must be understood to be expressly described any combination of any two features expressly described, even in the case in which the features are individually taken from the specific context, in which they may be joined to or combined with other, different features, taking account of the competence and knowledge of a person skilled in the art in the field who understands the possibility of functionally combining the features without it being necessary to functionally apply the other, different features. Unless specified otherwise, each and any element, member, means, system, component, object described and illustrated in the present description must be understood to be individually described and autonomously able to be modified and separated from and/or combined with each and any other element, member, means, system, component, object described and illustrated. The materials, forms and functions described and illustrated are not limitations on the present invention, but are only specified in order to enable a person skilled in the art to understand and carry out the invention according to preferred though non-exclusive embodiments.

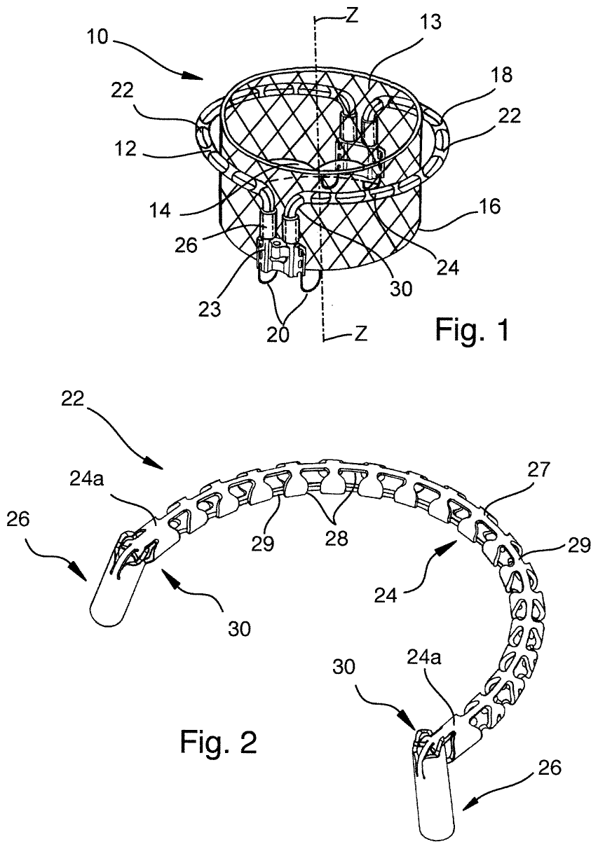

Now with reference to the drawings, FIG. 1 shows a valve prosthesis 10 for a heart valve. The valve prosthesis 10 is preferably used to replace the functionality of an atrioventricular valve, such as a mitral valve or a tricuspid valve. A valve prosthesis of this type and a method for the implantation thereof are described, for example, in WO 2021/014400, the entire content of which is incorporated herein by reference.

The valve prosthesis 10 comprises a prosthetic structure 12, for supporting and interfacing with the native valve. A group of flexible prosthetic leaflets 14 is arranged inside the prosthetic structure 12. The group of flexible prosthetic leaflets 14 is fixed to the prosthetic structure 12. The prosthetic structure 12 particularly comprises a prosthetic member 16, which is preferably tubular and which supports the prosthetic leaflets 14 which are arranged inside the tubular cavity. The prosthetic member 16 is surrounded by a retention member 18 which is intended to at least partially fix the prosthetic member 16 with respect to a heart tissue portion, in an implanted condition of the valve prosthesis 10. In the exemplary embodiment of the Figures, the retention member completely surrounds the prosthetic member 16 and forms a substantially closed ring, inside which the prosthetic member 16 is located. In variants which are not illustrated, the retention member can partially surround the prosthetic member, forming, for example, an open, C-shaped ring, or may form a plurality of turns, forming a helical winding around the prosthetic member.

The retention member 18 is stably secured to the prosthetic member 16 via connection members 20. The retention member 18 is configured so as to surround the native leaflets of the heart valve and to oppose an expansion thrust in a radial direction of the prosthetic member 16 in such a manner that, in the definitive implanted configuration, the native heart valves remain entrapped between the external surface of the prosthetic member 16 and the retention member 18 in order to stably anchor the valve prosthesis in the desired position inside the heart.

The prosthetic structure 12, as for each of the elements thereof, is configured so as to be collapsible without any repercussions on the safety and functionality of the valve prosthesis. Therefore, it is possible to temporarily reduce the radial spatial requirement of the valve prosthesis 10 in order to allow the introduction thereof inside the heart cavity through access ports with reduced opening, which are compatible with the surgery techniques involving minimal invasiveness, with transcatheter positioning and heart prosthesis implantation techniques and in particular with transcatheter techniques with transapical or transseptal access. Therefore, it is possible to insert the valve prosthesis 10 inside a catheter which has a low radial profile and which is able to convey the prosthesis inside the heart cavity near the implantation site, by means of access with minimal invasiveness, and to carry out at that location the deployment and implantation thereof, functionally replacing the native valve.

More specifically, the prosthetic member 16 is the portion of the prosthetic structure 12 which delimits a prosthetic conduit 13 for the passage of the blood through the device. Inside the prosthetic member 16, there are fixed the flexible prosthetic leaflets 14 which make the blood flow unidirectional inside the prosthetic conduit 13 in a manner parallel with the direction of a longitudinal axis Z-Z of the prosthetic conduit 13. The prosthetic member 16 is preferably a resilient structure which is radially collapsible with respect to the axis Z-Z and which, for example, also tends to expand as a result of resilient return to a diameter greater than the maximum diameter which maintains coaptation, that is to say, the contact, between the free edges of the closed prosthetic leaflets 14. The expansion of the prosthetic member 16 in the radial direction with respect to the axis Z-Z is limited by the retention member 18 in such a manner that the native leaflets of the heart valve are entrapped, as set out above, between the external surface of the prosthetic member 16 and the retention member 18. In a variant, the prosthetic member 16 does not expand as a result of resilient return, but is instead forced to expand, for example, by inflating a balloon therein, or with other techniques of the generally known type.

There extend outwards from the prosthetic member 16 the connection members 20 which also secure the retention member 18 to the prosthetic member 16 when it is still in a radially collapsed position with respect to the axis Z-Z inside the heart cavity.

The retention member 18 is the portion of the prosthetic structure 12 which opposes and limits the free expansion of the prosthetic member 16, preventing it from exceeding the maximum diameter which is compatible with the preservation of the coaptation between the prosthetic leaflets 14. In other words, the retention member 18 defines a perimeter of maximum expansion of the prosthetic member 16 and when the prosthetic member 16 reaches this perimeter it applies a force which is directed radially and which opposes the surrounding retention member in such a manner that the native leaflets of the heart valve remain entrapped and clamped between the external surface of the prosthetic member 16 and the retention member 18. The retention member 18 preferably has a geometry which is substantially annular and is peripherally substantially non-extensible, that is to say, it does not significantly modify the peripheral development thereof even when the prosthetic member 16 expands therein by applying a radial force to the retention member, with the interposition of the native leaflets. The retention member 18 may generally have any two-dimensional or three-dimensional form in accordance or not with the anatomy of the native atrio-ventricular valve. The retention member 18 may have a closed annular formation or also a partially open formation. In this second case, the retention member may be resiliently closable again or may comprise closure elements or may also maintain in the implanted configuration of the valve prosthesis 10 a partially open annular formation without the complete annular integrity being re-constituted.

The retention member 18 is composed of one or more components. Each component of the retention member 18 can be provided and introduced into the heart cavity separately from the prosthetic member 16 in order then to be connected stably thereto before the expansion thereof which makes the valve prosthesis stable in the final implanted configuration.

In a different embodiment of the invention, at least one component of the retention member 18 can be pre-connected at an end thereof to the body of the prosthesis 16 via a connection member 20, leaving the other end of the component free. In this manner, the at least one component of the retention member is introduced into the heart cavity together with the prosthetic member 16. Since it is introduced in a configuration with an end open, the at least one component of the retention member can be positioned so as to surround the native leaflets of the heart valve. Subsequently, the free end of the at least one component can be connected to the corresponding connection member 20 so as to re-constitute the implanted annular formation.

In the non-limiting exemplary embodiment of FIG. 1, the retention member 18 is formed by two mutually independent components 2 which are provided with connection members 26 which are engaged with the connection members 20 which extend from the prosthetic member 16 so as to secure it to the retention member 18. The components 22 preferably have an elongate form with respective main arcuate portions 24 which are substantially in the form of two half-rings which are located in the same plane, which is substantially perpendicular to the axis Z-Z. However, it is not excluded that the retention member may be formed by a single component, for example, a closed ring, a helix, a partially open ring or a ring which is initially open and can subsequently be closed again around the prosthetic member 16. In the same manner, it is not excluded that the retention member 18 may be composed of three or more components. If the retention member 18 is formed by a plurality of components, they can have a length, dimensions, for example, cross-section and/or thickness, and/or formation, for example, curvature and/or development in the plane or in space, which are identical to each other or different in all or in part, for all or only some of the components of the retention member 18.

In the non-limiting exemplary embodiment of FIG. 1, each component 22 has at the ends thereof two respective connection elements 26. Each connection element 26 preferably comprises and even more preferably is constituted by a hollow structure, particularly though in a non-limiting manner, substantially a hollow cylinder, even more preferably a hollow cylinder with a circular cross-section. The hollow structure of the connection element 26 is configured to allow the free passage of a guide wire.

The connection elements 26 have such a formation and dimensions as to allow a stable connection to the connection members 20. The connection members 20, which are preferably integral with the prosthetic member 16 and which protrude externally at the periphery thereof, are each provided to this end with a pair of pins 23. The pins 23 are preferably parallel with each other and sufficiently spaced apart from each other and are substantially aligned with the axis Z-Z of the prosthesis itself so as to allow connection to the connection elements 26, in particular by being inserted stably in the hollow structure thereof. Each pin 23 is also preferably hollow so as to allow the free sliding of a guide wire therein, preferably the same guide wire which can also slide inside the corresponding connection element 26.

In the final implanted configuration of the valve prosthesis 10 illustrated in FIG. 1, the connection elements 26 of each component 22 are orientated substantially perpendicularly to the main plane in which the main portion 24 is located. In other words, the connection elements 26 are parallel with the axis Z-Z. In this manner, the retention member 18 is generally, without prejudice to the connection elements 26, located in the same plane substantially perpendicular to the axis of the conduit 13 and therefore substantially parallel with the annular plane (that is to say, the plane defined by the annulus) of the native valve when the structural unit of the valve prosthesis 10 has been reconstituted. Both the connection elements 26 and the pins 23 which are present on the connection members 20 can be provided with mutual engagement members, such as plates or teeth or other surface discontinuities which are able to increase the friction or to block in a snap-fitting manner the connection between the pin and the hollow structure of the connection element, improving the stability of the connection.

The structure of each component 22 of the retention member 18 can have a passage for a guide wire along all or at least some of the length thereof, preferably a greater portion of the length thereof. In this manner, the positioning of the component 22 is simpler inside the ventricular cavity at the rear of the native valve leaflets. In fact, it is sufficient to arrange the guide wire, by means of known intervention techniques which are currently applied in clinical use, along the path which the desired positioning of the segment defines and to introduce this member by making it slide on the guide wire itself.

FIG. 2 shows to an enlarged scale one possible embodiment of the component 22 of the retention member 18 in the preferred form of an elongate component. This embodiment is particularly suitable for an implantation procedure which is carried out with transcatheter techniques. In particular, the embodiment of the component 22 illustrated in FIG. 2 refers to the case in which the retention member 18 is subdivided into two components. In any case, the following description can apply in the same manner to the case in which the retention member 18 comprises a different number of components 22, for example, a single component or more than two components.

For the sake of simplicity of illustration, FIG. 2 illustrates the single structural portion of the component 22 which forms the retention member 18 of the valve prosthesis 10. The structural portion is substantially obtained by a tubular element 27, on the wall of which apertures 28 have been formed with such dimensions and in such positions as to confer on the structure the desired resilient behaviour which can be anisotropic and variable from section to section in accordance with the position along the development of the component 22. In the embodiment shown in FIG. 2, the apertures 28 in the tubular element 27 define two opposite sets of ribs or backbones 29. One function of the backbones 29 is to provide the component with structural characteristics of longitudinal non-extensibility and rigidity to the deformations outside the plane, which are advantageous in the specific application. Naturally, apertures 28 of a different type from the one illustrated can be formed. For example, there can be formed apertures 28 which have a different geometry and/or different dimensions and/or a different position with respect to the one illustrated, for example, in order to provide the component with particular properties of resilient response. In an exemplary variant which is not illustrated, a single set of ribs 29 can be formed.

The structure of the component 22 which is formed in this manner can subsequently be shaped as schematically shown in FIG. 2, in which the main portion 24 and in particular the backbones 29, are curved in order to take up the geometry selected for the retention member 18. The connection elements 26 can be deflected at an angle with respect to the main portion 24. The most suitable material for this type of structural component, in which the requirement for optimum mechanical performance levels is accompanied by the requirement for behaviour in the resilient range including for great deformations, is in the range of the super-resilient alloys, for example, the equi-atomic alloy of nickel and titanium, which is commercially known by the name Nitinol. During the construction process, this structure can readily by obtained from a tube of the alloy selected, for example, Nitinol, in which there are formed the apertures 28, for example, by laser cutting. The final form, such as, for example, the form illustrated in FIG. 2, can be assigned by thermal processing operations which are applied to the workpiece secured inside a die. In the finished component 22, in order to reduce the risk of damage to the native leaflets in the connection zone with respect to the valve prosthesis, the structure can be completely or also only partially covered with tissue of a biological nature, for example, animal pericardium, or artificial nature, for example, PET textile or PTFE textile or with polymer material, for example, polyurethane or silicone-containing elastomer materials, or a combination of the two, for example, polymer material inside covered with textile.

FIG. 2 clearly shows the two connection elements 26 with a preferably cylindrical form which are deflected outside the main plane in which the main portion 24 of the component 22 is located. These connection elements 26 are the elements of the component 22 which are involved in the connection mechanism with respect to the prosthetic structure 12 of the valve prosthesis and which in particular are connected to the connection members 20 which as set out above are preferably integral with the prosthetic member 16 and protrude externally with respect to the periphery thereof. The connection elements 26 are joined in a pivotable manner to the main portion 24 of the structure of the component 22 by means of a pivotable connection, which is also referred to here as an articulation or hinge, and generally designated 30. The articulation 30 defines the connection which allows the mutual pivoting between a connection element 26 and a corresponding end 24a of the main portion 24. In the particular example of the Figures, each articulation 30 is defined in particular by a transition zone between the main portion 24 and each of the connection elements 26. In other words, in the preferred embodiments shown in the Figures, there is a material continuity between the end 24a of the main portion 24 of the component 22 and the corresponding connection element 26 because the articulation 30 is integrally formed in one piece with the structural portion of the main portion 24 and the connection element 26. The articulation 30 allows the connection element 26 to be articulated from a position, in which the longitudinal axis of the connection element 26 is substantially located in the same plane as the main portion 24 of the component 22, and is preferably substantially aligned with the adjacent end 24a of the main portion 24, to a position, such as the one illustrated in FIG. 2, in which the connection element 26 is deflected outside the main plane. The articulation 30 is formed in such a manner that the connection element 26 can be re-aligned with the plane of the main portion 24 but cannot be deflected beyond a predetermined angle, for example, but in a non-limiting manner, of approximately 90°, with respect to this plane. This solution prevents a greater deflection with respect to this predetermined angle between the main plane of the component 22 and the axis Z-Z of the prosthesis, once the component 22 is re-connected to the prosthetic member 16 of the valve prosthesis 10. In this manner, there are ensured both the continuity of the contact of the retention member 18 on the annulus of the native valve and the correct mutual alignment between the retention member 18 and the prosthetic member 16 when the valve prosthesis 10 is finally released.

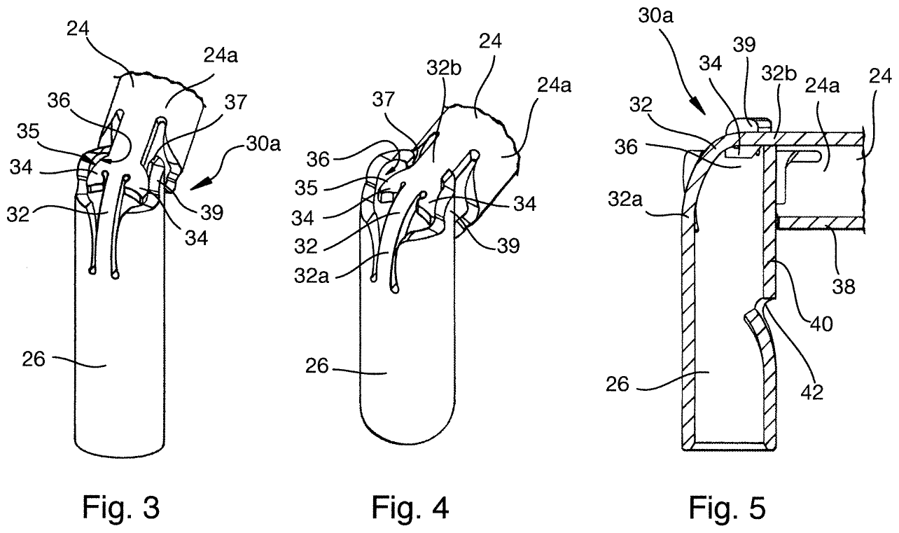

FIGS. 3 to 8 illustrate in greater detail a first embodiment of the articulation 30 which is indicated in this particular case as the articulation 30a. This embodiment is given by way of example, without it limiting in any way the generality of the invention. The articulation 30a is formed by means of cuts and apertures in the tubular element 27 which maintain the structural continuity of the component 22 through the flexible plate 32, which is positioned on the extrados of the articulation 30a, that is to say, in the region of the zone of the component 22 with the radius of curvature being greater when the articulation 30a is bent. In other words, the flexible plate 32 is the portion of the tubular element 27 which brings about, for any deflection angle of the connection element 26, the physical continuity between the main portion 24 and the connection element 26, via the corresponding articulation 30a.

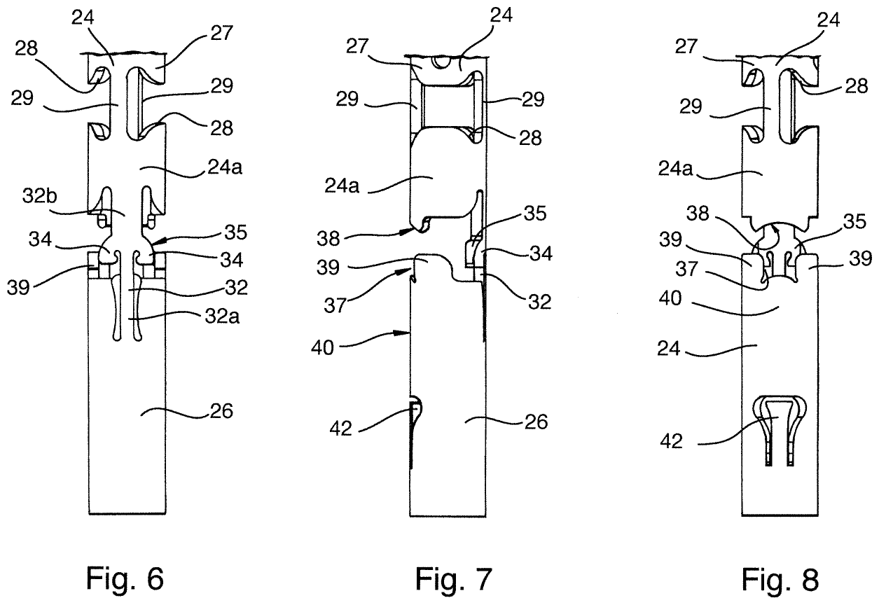

The plate 32 may have a different thickness and/or form in different portions of the length thereof, which define a different functional object of each section or portion of the plate itself. By way of non-limiting example, in the embodiment shown from FIG. 3 to FIG. 8, the flexible plate 32 which has a constant thickness corresponding to the thickness of the tubular element 27 in which it is formed, has a narrower section 32a, which is joined to the tubular section of the connection element 26, and a wider section 32b which is joined to the tubular section of the main portion 24. There extend laterally between the narrower section 32a and the wider section 32b of the flexible plate 32 two wings 34 which have arcuate edges 35 which are directed towards the wider section 32b. The narrower section 32a is the most readily deformable portion of the plate 32, that is to say, the portion which allows the flexion of the articulation 30a, with an acceptable level of stress of the material, so as to allow the connection element 26 to move from one to the other of the two extreme configurations, that is to say, the extended configuration, shown in FIGS. 6 to 8, which is co-planar with the main portion 24 and preferably aligned with the end 24a thereof, and the terminal deflection angle permitted by the geometry of the articulation 30a, and more generally by any articulation 30 described, in the region of which the articulation cannot be further deflected and becomes one with an angular rigidity which is substantially infinite. The portion with the two wings 34 has the objective of stably engaging with the connection element 26, engaging in a shaped recess 37 which is formed at the edge of the connection element 26 itself, which allows the wings 34 to be connected to the shoulders 39 which delimit this recess. To this end, the radius of the main portion of the profile 35 of the wings 34 is similar to the internal radius of the tubular element 27. The portion with the two wings 34 is in turn connected in a robust manner to the main portion 24 of the component 22 via the widest portion 32b of the plate 32, which is characterized by a greater resistant section which is therefore more suitable for supporting and transferring the cyclical stresses which are applied to the retention member 18 during the operation of the prosthesis 10.

In greater detail, when the articulation 30a is in the deflected configuration illustrated in FIGS. 4 and 5, which it can take up, for example, when the valve prosthesis 10 is definitively implanted, the arcuate edges 35 are intended to move into abutment and/or become interlocked against the corresponding internal wall 36 of the tubular element 27, in the region of the intrados of the articulation 30a. In this position, an end lip 38 of the main portion 24 is supported against the corresponding portion of the wall 40 of the connection element 26, which is located in a position diametrically opposite the plate 32. In the same rear wall 40 or more generally on the connection element 26, there can be formed one or more tongues 42 for engaging with one of the pins 23 which are present on the connection members 20, as described above. The axial positioning of the lip 38 with respect to the plate 32 defines the mechanical locking which brings about the bending limit of the articulation 30a, acting as an angular limit stop. The connection of the wings 34 to the shoulders 39 of the shaped recess 37, being supported against the internal wall 36 of the tubular element 27, cooperates with the width of the section of the most resistant portion 32b of the plate 32, the intrados of which moves in turn into abutment against the upper end of the wall 40 in order also to provide the articulation 30a with robust torsional stability. the portion Therefore, of the articulation 30a which is constituted by the portion with the wings 34 and the most resistant portion 32b of the plate 32 defines the resistant structural connection element between the main portion 24 of the component 22 and the connection element 26.

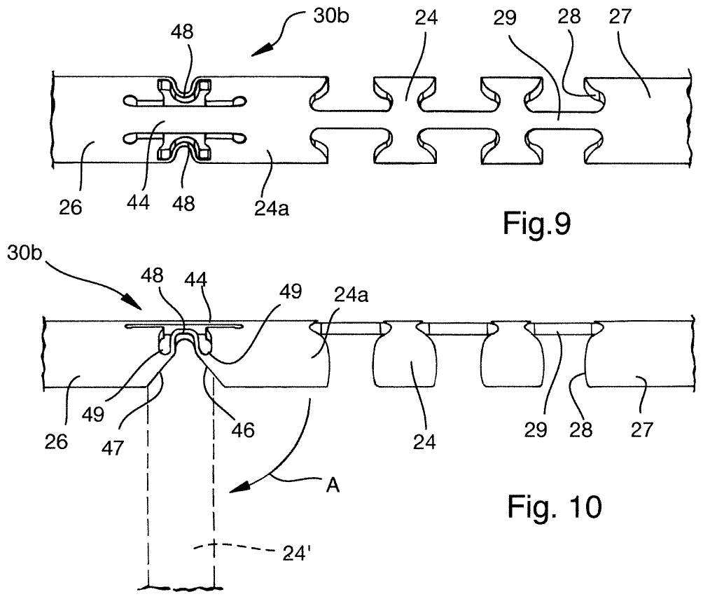

FIGS. 9 and 10 show a variant of an articulation 30b which is also given by way of non-limiting example. In the Figures, identical reference numerals correspond to identical elements with respect to those described above. In this case, the articulation 30b is also formed by means of cuts and apertures in the tubular element 27 which maintain the structural continuity of a flexible plate 44 which is positioned on the extrados of the articulation 30b, that is to say, in the region of the zone of the component 22 with the greatest radius of curvature when the articulation 30b is bent. In other words, the flexible plate 44 is the portion of the tubular element 27 which integrally joins the main portion 24 to the connection element 26 of the corresponding articulation 30b.

In this variant, the flexible plate 44 preferably has the same width or thickness over the entire extent thereof from the end which is joined to the tubular section of the connection element 26 as far as the end which is joined to the tubular section of the main portion 24. In a variant which is not illustrated, the flexible plate 44 is shaped with portions of different widths or thicknesses in order to confer a flexion behaviour thereon with non-homogeneous curvature, in accordance with the methods and the characteristics indicated in relation to the articulation 30a described above.

At the side opposite the flexible plate 44, the tubular section of the connection element 26 and the tubular section of the main portion 24 are cut in a specular manner in the manner of a flute mouthpiece, that is to say, obliquely with respect to the axis of the tubular sections, so as to define two opposite support edges 46, 47. The two support edges 46, 47 are joined to each other near the flexible plate 44 by a narrow flexible section 48 which is obtained by enlarging with slots 49 the longitudinal cuts which define and isolate the flexible plate 44 from the remainder of the wall of the tubular element 27.

When the articulation 30b is bent from the extended configuration, illustrated with a solid line in FIG. 10, to the configuration taken up in the implanted prosthesis 10, wherein the main portion 24 is rotated in accordance with the arrow A in order to be brought into the dashed position 24′, the two support edges 46, 47 move into abutment one against the other, defining the mechanical locking action which limits the bending of the articulation 30b, acting as an angular limit stop. At the same time, the two flexible sections 48, which are laterally spaced apart on the transverse rotation axis of the main portion 24 with respect to the connection element 26, provide the articulation 30b with a robust torsional stability. The transverse dimension of the section of the flexible plate 44 which at least partially opposes the pivoting actions between the main portion 24 and the connection element 26 also contributes to this.

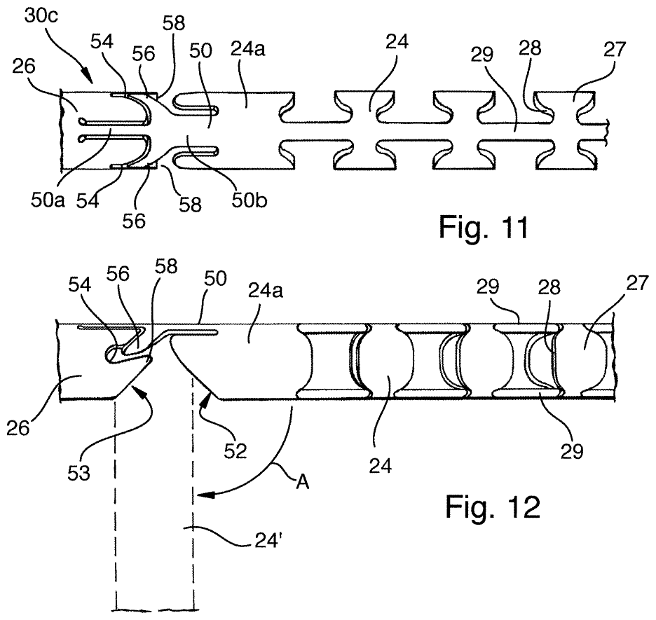

FIGS. 11 and 12 show another variant of an articulation 30c, which is also given by way of non-limiting example. In the Figures, identical reference numerals correspond to elements identical to those described above. In this case, the articulation 30c is also constructed by cuts and apertures in the tubular element 27 which maintain the structural continuity via a flexible plate 50 which is positioned on the extrados of the articulation 30c, that is to say, in the zone of the component 22 with the greatest radius of curvature when the articulation 30c is bent. In other words, the flexible plate 50 is the portion of the tubular element 27 which forms, for any deflection angle of the connection element 26, the physical continuity between the main portion 24 and the connection element 26 of the corresponding articulation 30c.

In this variant, the flexible plate 50 may have the same width or thickness over the entire extent thereof or may be shaped with sections of different widths, thicknesses or in any case with different resistances in order to confer thereon a flexion behaviour with a non-homogeneous curvature so as to take up the behaviour and characteristics described above with reference to the articulation 30a. The example of the Figures shows that the flexible plate 50 comprises a section 50a which is narrower and longer than a section 50b, which confer on the plate 50 a different flexion behaviour.

At the opposite side to the flexible plate 50, the tubular section of the connection element 26 and the tubular section of the main portion 24 are cut in a specular manner in the manner of a flute mouthpiece, that is to say, obliquely with respect to the axis of the tubular sections, so as to define two support edges 52, 53. The support edge 52 which is formed at the end of the main portion 24 extends until being connected to the edge of the cut 54 which defines the portion of the flexible plate 50 which extends from the main portion 24. The support edge 53 extends from the two portions of the connection element 26 until being connected to two lateral recesses 54, forming two respective peaks 58. In the region of the recesses 54, there extend from both sides of the flexible plate 50 two wings 56 which project towards the connection element 26 and face each other in the direction of the recesses 54.

When the articulation 30c is bent from the extended configuration illustrated with a solid line in FIG. 12, in order to reach the configuration taken up in the implanted prosthesis 10, wherein the main portion 24 is rotated in accordance with the arrow A in order to be brought into the dashed position 24′, the two support edges 52, 53 move into abutment against each other, defining the mechanical locking action which limits the bending of the articulation 30c, acting as an angular limit stop. The design of the wings 56 is such that, during this rotation of the articulation 30c, they slide inside the recesses which are defined by the edge of the cut 54, but in any case remain supported on the peaks 58, distributing over a greater resistant section the stress which during operation of the implanted prosthesis 10, is transferred from the main portion 24 to the connection element 26 of the component 22. In this manner, the wings 56 contribute significantly to the robustness, the fatigue resistance and the stability, including torsional stability, of the articulation 30c. The flexible plate 50 which also opposes the pivoting actions between the main portion 24 and the connection element 26 also contributes to the overall robustness of the articulation 30c.

In another variant of this embodiment of the invention, the design of the articulation can be such that, in the extended position illustrated with a solid line in FIG. 12, the ends of the wings 56, by interfering with the respective peaks 58, prevent the articulation 30c from bending in the opposite direction to that of the arrow A, thereby stabilizing a securing action which prevents errors during assembly or installation of the component 22 and therefore of the retention member 18 of the valve prosthesis 10 or which prevents the application of an excessive stress of the plate 50 itself, with an excessive bending of the articulation in the direction without the angular mechanical locking means.

In another variant of this embodiment of the invention, the design of the wings 56 and the recess 54 is such that, in the configuration which the articulation 30c takes up in the implanted prosthesis 10, the wings 56 are locked in the respective recesses 54, blocking the articulation 30c in the bent position, providing them equally with an increase in robustness and fatigue resistance, but also opposing the re-expansion of the articulation 30c into the extended position.

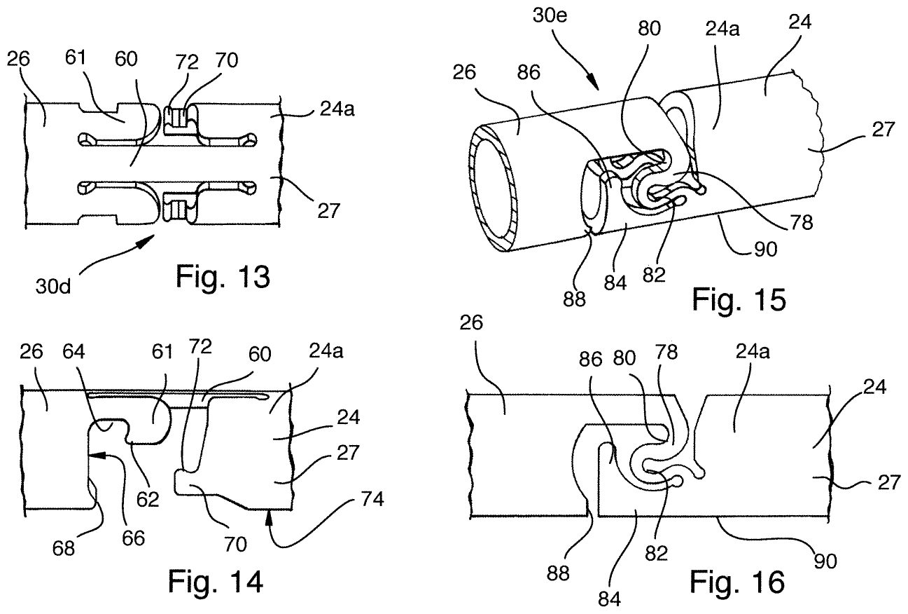

FIGS. 13 and 14 show another variant of an articulation 30d which is also given by way of non-limiting example. In the Figures, identical reference numerals correspond to elements identical to those described above. In this case, the articulation 30d is also constructed by means of cuts and apertures in the tubular element 27 which maintain the structural continuity of a flexible plate 60 which is positioned on the extrados of the articulation 30d, as defined above. In this case, the flexible plate 60 is also the portion of the tubular element 27 which forms, for any deflection angle of the connection element 26, the physical continuity between the main portion 24 and the connection element 26 of the corresponding articulation 30d.

In this variant, the flexible plate 60 has the same width or thickness over the entire extent thereof. Naturally, in this case the flexible plate 60 can also be shaped with sections of different widths or thicknesses in order to confer thereon a flexion behaviour with a non-homogeneous curvature so as to take up the behaviour and characteristics described above with reference to the articulation 30a. At the opposite side to the flexible the tubular section of the connection element 26 is shaped so as to have two projections 61 which each define a hook 62 at each side of the flexible plate 60. The projections 61 define respective recesses 64 which are connected to an edge 66 of the connection element 26. In a diametrically opposed position to the flexible plate 60, the edge 66 has a prominent support lip 68.

The end of the main portion 24 is shaped with two projections 70 which are each provided with a hook 72 with such a profile that, in the position bent through approximately 90° of the articulation 30d (not illustrated), it engages with the respective hook 62, becoming introduced into the recess 64 thereof. In this bent position, the lip 68 moves into a state supported against a portion of rear wall 74 of the main portion 24, which is located in a position diametrically opposite the flexible plate 60. The support between the lip 68 and the rear wall 74 forms the mechanical locking which defines the bending limit of the articulation 30d, acting as an angular limit stop. The engagement of the projections 70 with the hooks 62 provides the articulation 30d with a robust torsional solidity and better distribution of the loads which are transferred to the connection element 26 by the main portion 24 of the component 22.

It is possible to modify the design of the projections 61 and 70 and the profiles of the recesses and hooks 62 and 72 so as to form different methods of engagement between the two elements of the articulation 30d and/or to increase the capacity for support given by the connection of the surfaces.

FIGS. 15 and 16 show another variant of an articulation 30e which is also given by way of non-limiting example. In the Figures, identical reference numerals correspond to elements identical to those described above. In this case, the articulation 30e is also formed by means of cuts and apertures in the tubular element 27. In this embodiment, there is not provided a flexible articulation plate and the structural continuity between the main portion 24 and the connection element 26 is provided by two S-shaped wall sections 78 which are opposite, symmetrical with respect to a longitudinal plane which is orthogonal to the rotation axis of the articulation 30e. Each S-shaped wall section 78 is formed so as to form a first loop 80 on the connection element 26. After forming the first loop 80, the S-shaped wall section 78 is bent in order to form a second loop 82 at the end of which the S-shaped wall section 78 is joined to the main portion 24. There extends from the main portion 24 an extension 84 which forms two lateral fingers 86 which are shaped so as to each be connected in one of the first loops 80 of the S-shaped wall section 78 in the position bent through approximately 90° of the articulation 30e (not illustrated). In the bent position, a support lip 88 of the connection element 26 is supported against a portion of the rear wall 90 of the main portion 24, which is located at the side of the extension 84. The support between the lip 88 and the rear wall 90 forms the mechanical locking action which defines the bending limit of the articulation 30e, acting as an angular limit stop. The engagement of the fingers 86 in the first loops 80 provides the articulation 30e with a robust torsional solidity and particularly significantly unloads the deformable portion of the articulation in terms of the fatigue loads which the main portion 24 of the component 22 transfers to the connection element 26.

With a suitable design of the lateral fingers 86 and the profile of the loop 80, for example, with geometric discontinuities which generate undercuts, it is possible to make the articulation irreversible so that the permanent mechanical connection between the two elements prevents the return of the articulation from the bent position to the extended position illustrated.

However, notwithstanding the fact that in FIGS. 15 and 16 there have been conventionally indicated on the right the main portion 24 which is provided with the fingers 86 and on the left the connection element 26, on which the first loops 80 are formed, the possibility is not excluded of inverting the articulation 30e, forming the fingers 86 on the connection element 26 and the first loops 80 on the main portion 24.

Naturally, the principle of the invention remaining the same, the forms of embodiment and details of construction may be varied widely with respect to those described and illustrated without thereby departing from the scope of the present invention.

1. A valve prosthesis for a heart valve, comprising:
a prosthetic member which supports prosthetic leaflets which are able to reproduce functionality of valve leaflets of a native heart valve; and
a retention member at least partially fixing the prosthetic member with respect to a portion of heart tissue in an implanted condition of the valve prosthesis, wherein the retention member comprises at least one component having a main portion joined at an end thereof to at least one connection element via a pivotable connection for connecting the retention member to the prosthetic member, the connection element and the main portion being connected in a pivotable manner with respect to each other so as to pivot from a first position to a second position, in which the connection element is deflected with respect to the end of the main portion, the component further comprising connection members to stably connect the main portion and the connection element in the second position so as to limit the stresses to which the pivotable connection is subjected under operating conditions of the valve prosthesis.

2. The valve prosthesis according to claim 1, wherein the pivotable connection between the connection element and the main portion comprises at least one flexible section configured to maintain a physical continuity between the main portion and the connection element.

3. The valve prosthesis according to claim 2, wherein the at least one flexible section is arranged at an extrados of the pivotable connection.

4. A valve prosthesis for a heart valve, comprising:
a prosthetic member which supports prosthetic leaflets which are able to reproduce functionality of valve leaflets of a native heart valve; and
a retention member at least partially fixing the prosthetic member with respect to a portion of heart tissue in an implanted condition of the valve prosthesis, wherein the retention member comprises at least one component having a main portion joined at one end thereof to at least one connection element via a pivotable connection to connect the retention member to the prosthetic member, the connection element and the main portion being connected in a pivotable manner with respect to each other to pivot from a first position to at least one second position, in which the connection element is deflected with respect to the end of the main portion, and wherein the pivotable connection between the connection element and the main portion comprises a flexible section in a region of an extrados of the pivotable connection to maintain a physical continuity between the main portion and the connection element.

5. The valve prosthesis according to claim 2, wherein the flexible section comprises a portion with less resistance to provide a predetermined flexibility for the pivotable connection during movement from the first position to the second position, and a portion with greater resistance which defines a resistance of the pivotable connection to the stresses to which the pivotable connection is subjected in the implanted condition of the valve prosthesis.

6. The valve prosthesis according to claim 5, wherein the flexible section comprises a flexible plate with a first portion having a first cross-section and a second portion having a second cross-section greater than the first cross-section, the first portion comprising the portion with less resistance and the second portion comprising the portion with greater resistance.

7. The valve prosthesis according to claim 2, wherein the flexible section is non-extensible.

8. The valve prosthesis according to claim 2, wherein the flexible section is integrally formed with the component and forms a single piece with the main portion and the connection element.

9. The valve prosthesis according to claim 2, wherein the flexible section is arranged in a region of an extrados of the pivotable connection between the connection element and the main portion.

10. The valve prosthesis according to claim 3, wherein the component further comprises connection members to stably connect the main portion and the connection element in the second position to limit the stresses to which the pivotable connection is subjected under operating conditions of the valve prosthesis, wherein the connection members comprise wings which project laterally from the flexible section and are configured for engagement in at least one corresponding cavity formed in the connection element.

11. The valve prosthesis according to claim 1, wherein the at least one connection element is resiliently connected to the main portion of the component that a thrust is applied to the component in a direction of the first position.

12. The valve prosthesis according to claim 1, wherein the pivotable connection is formed by cuts and apertures in a tubular element, the cuts and apertures maintaining a structural continuity between the connection element and the main portion of the component.

13. The valve prosthesis according to claim 12, wherein the tubular element integrally defines the component.

14. The valve prosthesis according to claim 1, wherein the second position corresponds to a position of maximum deflection which is taken up at least in the implanted condition of the prosthesis and in which the connection element is deflected with respect to the end of the main portion by a maximum angle beyond which the connection element cannot be further deflected.

15. A method for implanting a valve prosthesis for a heart valve, comprising the steps of:
providing a prosthetic member which supports prosthetic leaflets able to reproduce a functionality of valve leaflets of a native heart valve;
providing a retention member to at least partially fix the prosthetic member with respect to a portion of heart tissue in an implanted condition of the valve prosthesis, the retention member comprising at least one component having a main portion joined at one end thereof to at least one connection element via a pivotable connection for connecting the retention member to the prosthetic member;
configuring the connection element and the main portion of the retention member in a first position, in which the connection element and the main portion of the component take up a configuration with a minimal spatial requirement compatible with-the passage thereof in a catheter;
introducing the component into a heart cavity by passage in a catheter;
configuring the connection element and the main portion of the retention member in a second position, in which the connection element is deflected with respect to the end of the main portion;
stably connecting, with connection members, the main portion and the connection element in the second position to limit the stresses to which the pivotable connection is subjected under operating conditions of the valve prosthesis;
introducing the prosthetic member into the heart cavity; and
at least partially fixing the prosthetic member with the retention member with respect to a portion of heart tissue in order to achieve an implanted condition of the valve prosthesis.
